(19) United States
(12) Patent Application Publication
(10) Pub. No.: US 2003/0103929 A1
(43) Pub. Date: Jun. 5, 2003

(54) Cosmetic compositions containing a methacrylic acid copolymer, a silicone and a cationic polymer, and uses thereof

(76) Inventor: Mireille Maubru, Chatou (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/237,679

(22) Filed: Sep. 10, 2002

(30) Foreign Application Priority Data
Sep. 11, 2001 (FR) 01 11742

(51) Int. Cl. A61K 7/06

(52) U.S. Cl. 424/70.16

(57) ABSTRACT

Cosmetic compositions comprising, in a cosmetically acceptable medium, at least one copolymer of methacrylic acid and a C₁-C₄ alkyl acrylate, at least one polymer chosen from cationic and amphoteric polymers whose cationic charge density is greater than or equal to 3.5 meq/g, and at least one silicone chosen from polydialkylsiloxanes containing dimethylsilanol end groups. These compositions can be used for washing and/or conditioning keratin materials such as the hair or the skin.

The present invention relates to novel cosmetic compositions comprising, in a cosmetically acceptable medium, at least one methacrylic acid/C₁-C₄ alkyl acrylate copolymer, at least one polymer chosen from cationic and amphoteric polymers and at least one particular silicone.

It is well known that hair, which has been sensitized (i.e. damaged and/or embrittled) to varying degrees under the action of atmospheric agents or under the action of mechanical or chemical treatments, such as dyes, bleaches and/or permanent-waving, can often be difficult to disentangle and to style, and may lack softness.

It has already been recommended to use conditioners, such as cationic polymers or silicones, in compositions for washing or caring for keratinous material such as the hair, in order to be able to disentangle the hair and to give it softness and flexibility. However, at least one cosmetic advantage mentioned above is also unfortunately accompanied, on dried hair, by certain cosmetic effects considered as being undesirable, i.e. lankness of the hairstyle (lack of lightness of the hair) and lack of smoothness (hair not uniform from the root to the tip).

In addition, the use of cationic polymers for this purpose can have various drawbacks. On account of their high affinity for the hair, some of these polymers can become deposited thereon to a large extent during repeated use, and can lead to adverse effects such as an unpleasant, laden (charged or loaded) feel, stiffening of the hair and interfibre adhesion, which has an effect on styling. These drawbacks can be accentuated in the case of fine hair, which lacks liveliness and body.

Functionalized silicones are generally used in shampoo compositions as conditioners for improving the softness, feel and disentangling of the hair. However, it has been found that these silicones can lead to the formation of an unattractive layer at the surface of the shampoo, which can be harmful to the performance of the shampoo. To avoid the appearance of this phenomenon, stabilizers such as crosslinked acrylic polymers of the Carbopol type are frequently used. However, these stabilizers can have the drawback of reducing the cosmetic performance of shampoos, such as by making the hair more laden and coarser.

In summary, it is found that the current cosmetic compositions containing cationic or amphoteric polymers are not entirely satisfactory.

Certain cosmetic compositions, such as detergents, have been disclosed, containing a copolymer of methacrylic acid and of an alkyl acrylate, as a stabilizer or suspension agent for water-insoluble ingredients, for instance silicones or fatty substances. Such compositions have been described, for example, in patent application WO 01/76552. The foam quality and the cosmetic properties obtained with these compositions are still not sufficiently satisfactory.

The inventor has now discovered that the combination of at least one copolymer chosen from crosslinked and non-crosslinked methacrylic acid/C₁-C₄ alkyl acrylate copolymers, at least one polymer chosen from cationic and amphoteric polymers whose cationic charge density is greater than or equal to 3.5 meq/g and at least one particular silicone, makes it possible to overcome at least one of these drawbacks.

It has been found that the use of the said acrylic copolymer in the compositions of the present invention can produce on keratin materials, such as the hair, good cosmetic properties, for example, as regards the lightness, softness, smooth feel, suppleness and manageability of dried hair. It has also been found that, with the compositions of the invention, dried hair that looks generally smoother can be obtained.

Moreover, the compositions according to the invention can be stable and can have an attractive visual appearance. The usual properties (appearance, consistency, foam abundance, elimination of foam) can be satisfactory.

The compositions of the invention, when applied to the skin, such as in the form of a bubble bath or a shower gel, can give an improvement in the softness of the skin.

Thus, according to the present invention, novel cosmetic compositions are now proposed, comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

Another aspect of the invention is also the composition as defined above, as well as methods, to give the hair at least one of sheen, lightness, softness, a smooth feel and suppleness.

Another aspect of the invention relates to a process for treating a keratin material, such as the hair, characterized in that it comprises applying to the keratin material a cosmetic composition according to the invention.

According to the present invention, the expression “keratin materials” means the hair, the eyelashes, the eyebrows, the skin, the nails, mucous membranes or the scalp.

Another aspect of the invention relates to the addition of a copolymer chosen from crosslinked and non-crosslinked copolymers of methacrylic acid and of a C₁-C₄ alkyl acrylate in, or for the manufacture of, a cosmetic composition comprising at least one polymer chosen from cationic and amphoteric polymers whose cationic charge density is greater than or equal to 3.5 meq/g and at least one silicone as defined above.

Various aspects of the invention will now be detailed. All the meanings and definitions of the compounds used in the present invention given below are valid for all the aspects of the invention.

One of the characteristics of the invention is the presence of at least one copolymer chosen from crosslinked and non-crosslinked copolymers of methacrylic acid and of a C₁-C₄ alkyl acrylate.

The methacrylic acid residue is, for example, present in amounts ranging from 20% to 80% by weight and further, for example, from 25% to 70% by weight and even further, for example, from 35% to 60% by weight relative to the total weight of the copolymer.

The alkyl acrylate residue is, for example, present in amounts ranging from 15% to 80% by weight and further, for example, from 25% to 75% by weight and even further, for example, from 40% to 65% by weight relative to the total weight of the copolymer. It can be chosen from methyl acrylate, ethyl acrylate and butyl acrylate residues. This copolymer is, for example, partially or totally crosslinked with at least one standard crosslinking agent. The crosslinking agents are, for example, polyunsaturated compounds, such as ethylenically polyunsaturated compounds. These compounds are, for example, chosen from polyalkenyl ethers of sucrose and of polyols, diallyl phthalates, divinylbenzene, allyl (meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, and derivatives of castor oil and of polyols manufactured from unsaturated carboxylic acids.

Crosslinking agents that may also be used include, for example, unsaturated monomer compounds comprising a reactive group capable of reacting with an unsaturation to form a crosslinked copolymer.

The content of the crosslinking agent generally ranges, for example, from 0.01% to 5% by weight and further, for example, from 0.03% to 3% by weight and even further, for example, from 0.05% to 1% by weight relative to the total weight of the copolymer.

According to one embodiment of the present invention, the copolymer of the invention may be, for example, in the form of a dispersion in water. The number-average size of the particles of copolymer in the dispersion is, for example, from 10 to 500 nm, as measured by appropriate means known to those skilled in the art, and further, for example, from 20 to 200 nm and even further, for example, from 50 to 150 nm.

These copolymers are described, for example, in patent application WO 01/76552.

Use can be made, for example, of the methacrylic acid/ethyl acrylate crosslinked copolymer in the form of an aqueous 30% dispersion manufactured and sold under the name Carbopol Aqua SF-1 by the company Noveon.

The copolymer concentration is generally, for example, from 0.01% to 10% by weight relative to the total weight of the composition and further, for example, from 0.1% to 5% by weight relative to the total weight of the composition.

The silicones that may be used in accordance with the invention are, for example, insoluble in the composition and may be in the form of oils, waxes or gums.

The silicones are defined in greater detail in Walter Noll's publication “Chemistry and Technology of Silicones” (1968) Academic Press.

According to the invention, all the silicones may also be used in the form of emulsions, nanoemulsions or microemulsions.

The silicones according to the invention are, for example, chosen from polydi(C1-C4)alkylsiloxanes, among which mention may be made, for example, of polydimethylsiloxanes comprising dimethylsilanol end groups (Dimethiconol according to the CTFA name) and, for example, having a viscosity of from 5×10⁻⁶ to 2.5 m²/s at 25° C., and further, for example, of from 1×10⁻⁵ to 1 m²/s at 25° C. The viscosity of the silicones is measured, for example, at 25° C. according to ASTM standard 445 Appendix C.

Among these silicones comprising dimethylsilanol end groups, mention may be made in a non-limiting manner of

the products sold by Dow Corning under the name Dow Corning 5-0309 and DC1784

the products sold by Witco under the name TP512 and Silicone Emulsion LE467,

the products sold by Bayer Silicone under the name Baysilone Fluid TO-5, TO-9 and TO-7, and

the products sold by Ballu Chimie under the names DP98/004 and DP98/005.

The at least one silicone is, for example, used in an amount ranging from 0.01% to 20% by weight relative to the total weight of the composition. Further, for example, this amount ranges from 0.05% to 15% by weight, and even further, for example, from 0.1% to 10% by weight relative to the total weight of the composition.

The cationic polymers that may be used according to the invention have a cationic charge density of greater than or equal to 3.5 meq/g and, for example, from 3.5 to 8.5 meq/g. The charge density may be determined according to the Kjeldahl method. It is generally measured at a pH of the order of 3 to 9.

The cationic polymers that may be used in accordance with the present invention may be chosen from any of those already known as improving the cosmetic properties of the hair, for example, those described in patent application EP-A-0 337 354 and in French patent applications FR-A-2 270 846, 2 383 660, 2 598 611, 2 470 596 and 2 519 863 and having a cationic charge density of greater than or equal to 3.5 meq/g.

As used herein, “cationic polymer” refers to any polymer chosen from polymers comprising at least one cationic groups and polymers comprising at least one group that may be ionized into cationic groups.

The cationic polymers may, for example, be chosen from those comprising units comprising primary, secondary, tertiary and/or quaternary amine groups that may either form part of the main polymer chain, or may be borne by a side substituent that is directly attached to the main chain.

The cationic polymers used generally have a number-average or weight-average molar mass ranging from 500 to 5×10⁶ and, for example, from 1 to 3×10⁶.

Among the cationic polymers that may be mentioned are, for example, polymers of polyamine, polymers of polyamino amide and polymers of polyquaternary ammonium. These polymers are known in the art.

The polymers of polyamine, polymers of polyamino amide and polymers of polyquaternary ammonium that may be used in accordance with the present invention, and that may be mentioned, for example, are those described in French Patent Nos. 2 505 348 and 2 542 997. Among these polymers, mention may be made of:

(1) homopolymers or copolymers derived from acrylic or methacrylic esters or amides and comprising at least one of the units of the following formulae:

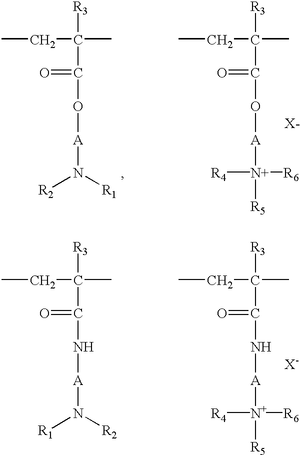

in which:

R₃, which may be identical or different, is chosen from a hydrogen atom and a CH₃ radical;

A, which may be identical or different, is chosen from linear and branched alkyl groups of 1 to 6 carbon atoms, for example, 2 or 3 carbon atoms, and hydroxyalkyl groups of 1 to 4 carbon atoms;

R₄, R₅ and R₆, which may be identical or different, are chosen from alkyl groups comprising from 1 to 18 carbon atoms and benzyl radicals and such as alkyl groups comprising from 1 to 6 carbon atoms;

R₁ and R₂, which may be identical or different, are chosen from a hydrogen atom and alkyl groups comprising from 1 to 6 carbon atoms, and such as methyl and ethyl;

X⁻ is an anion derived from an inorganic or organic acid, such as a methosulphate anion or an anion chosen from halides such as chloride and bromide.

Copolymers of family (1) can also comprise at least one unit derived from comonomers, which may be chosen from the family of acrylamides, methacrylamides, diacetone acrylamides, acrylamides and methacrylamides substituted on the nitrogen with at least one group chosen from lower (C₁-C₄) alkyls, acrylic acids or esters thereof, or methacrylic acids or esters thereof, vinyllactams, such as vinylpyrrolidone and vinylcaprolactam, and vinyl esters.

Thus, among these copolymers of family (1), mention may be made of:

the copolymers of acrylamide and of dimethylaminoethyl methacrylate quaternized with dimethyl sulphate or with a dimethyl halide,

the copolymers of acrylamide and of methacryloyloxyethyltrimethylammonium chloride described, for example, in patent application EP-A-080 976,

the copolymers of acrylamide and of methacryloyloxyethyltrimethylammonium methosulphate,

quaternized or non-quaternized vinylpyrrolidone/dialkylaminoalkyl acrylate or methacrylate copolymers. These polymers are described in detail in French Patent Nos. 2 077 143 and 2 393 573,

dimethylaminoethyl methacrylate/vinylcaprolactam/vinylpyrrolidone terpolymers,

vinylpyrrolidonelmethacrylamidopropyldimethylamine copolymers, and

quaternized vinylpyrrolidone/dimethylaminopropylmethacrylamide copolymers.

(2)cationic polysaccharides, such as celluloses and cationic galactomannan gums. Among the cationic polysaccharides that may be mentioned, for example, are cellulose ether derivatives comprising quaternary ammonium groups, cationic cellulose copolymers or cellulose derivatives grafted with a water-soluble quaternary ammonium monomer, and cationic galactomannan gums.

The cellulose ether derivatives comprising quaternary ammonium groups are, for example, described in French Patent No.1 492 597.

The cationic cellulose copolymers or cellulose derivatives grafted with a water-soluble quaternary ammonium monomer are described, for example, in U.S. Pat. No. 4,131,576, such as hydroxyalkylcelluloses, for instance hydroxymethylcelluloses, hydroxyethylcelluloses and hydroxypropylcelluloses grafted, for example, with a salt chosen from methacryloylethyltrimethylammonium salts, methacrylamido-propyltrimethylammonium salts and dimethyldiallylammonium salts.

The cationic galactomannan gums are described, for example, in U.S. Pat. Nos. 3,589,578 and 4 031 307, such as guar gums comprising trialkylammonium cationic groups. For example, guar gums modified with a salt (e.g. chloride) of 2,3-epoxypropyltrimethylammonium may be used.

(3)polymers comprising piperazinyl units and divalent alkylene or hydroxyalkylene radicals comprising straight or branched chains, optionally interrupted by at least one atom chosen from oxygen, sulphur and nitrogen atoms or by at least one ring chosen from aromatic and heterocyclic rings, as well as at least one of the oxidation and/or quaternization products of these polymers. Such polymers are described, for example, in French Patent Nos. 2 162 025 and 2 280 361;

(4)water-soluble polyamino amides prepared, for example, by polycondensation of an acidic compound with a polyamine; these polyamino amides being crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound, which is reactive with a bis-halohydrin, a bis-azetidinium, a bis-haloacyldiamine, a bis-alkyl halide, an epihalohydrin, a diepoxide or a bis-unsaturated derivative. The crosslinking agent can be used in proportions ranging from 0.025 to 0.35 mol per amine group of the polyamino amide. These polyamino amides can be alkylated or, if they comprise at least one tertiary amine function, they can be quaternized. Such polymers are described, for example, in French Patent Nos. 2 252 840 and 2 368 508;

(5)polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and, for example, methyl, ethyl or propyl. Such polymers are described, for example, in French Patent No. 1 583 363.

Among these derivatives, mention may be made, for example, of the adipic acid/dimethylaminohydroxypropyl/diethylenetriamine polymers.

(6)polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 8 carbon atoms.

The molar ratio between the polyalkylene polyamine and the dicarboxylic acid may range, for example, from 0.8:1 to 1.4:1; the polyamino amide resulting therefrom may be reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2 961 347.

(7)cyclopolymers of alkyldiallylamine or of dialkyldiallylammonium, such as the homopolymers or copolymers comprising, as main constituent of the chain, at least one unit corresponding to formula (I) or (I′):

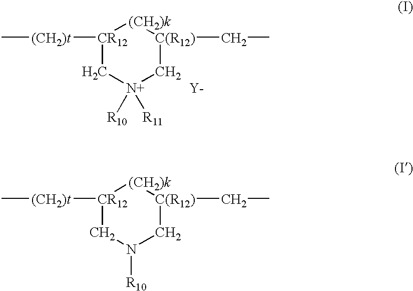

in which k and t are equal to 0 or 1, the sum k+t being equal to 1;

R₁₂ is chosen from a hydrogen atom and a methyl radical;

R₁₀ and R₁₁, which may be identical or different, are chosen from alkyl groups comprising from 1 to 6 carbon atoms, hydroxyalkyl groups in which the alkyl group, for example, comprises from 1 to 5 carbon atoms, and lower C₁-C₄ amidoalkyl groups, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate and phosphate. These polymers are described, for example, in French Patent No. 2 080 759 and in its Certificate of Addition 2 190 406.

In one embodiment, R₁₀ and R₁₁, which may be identical or different, for example, are chosen from alkyl groups comprising from 1 to 4 carbon atoms.

Among the polymers defined above, mention may be made, for example, of the dimethyldiallylammonium chloride homopolymer sold under the name “Merquat 100” by the company Nalco (its homologues of low weight-average molecular mass) and copolymers of diallyldimethylammonium chloride and of acrylamide

(8)quaternary diammonium polymers comprising repeating units corresponding to the formula (II):

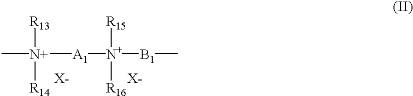

in which:

R₁₃, R₁₄, R₁₅ and R₁₆, which may be identical or different, are chosen from aliphatic, alicyclic and arylaliphatic radicals comprising from 1 to 20 carbon atoms and lower hydroxyalkylaliphatic radicals, or R₁₃, R₁₄, R₁₅ and R₁₆, together or separately, constitute, with the nitrogen atoms to which they are attached, heterocycles optionally comprising a second hetero atom other than nitrogen, or R₁₃, R₁₄, R₁₅ and R₁₆ are chosen from linear and branched C₁-C₆ alkyl radicals substituted with at least one group chosen from nitrile, ester, acyl and amide groups and groups of formulae —CO—O—R₁₇-D and —CO—NH—R₁₇-D wherein R₁₇ is chosen from alkylene groups and D is chosen from quaternary ammonium groups;

A₁ and B₁, which may be identical or different, are chosen from linear and branched, saturated and unsaturated polymethylene groups comprising from 2 to 20 carbon atoms. The polymethylene groups may comprise, linked to or intercalated in the main chain, at least one entity chosen from aromatic rings, oxygen and sulphur atoms and sulphoxide, sulphone, disulphide, amino, alkylamino, hydroxyl, quaternary ammonium, ureido, amide and ester groups; and

X⁻ is an anion chosen from anions derived from inorganic acids and organic acids.

A₁, R₁₃ and R₁₅ may optionally form, with the two nitrogen atoms to which they are attached, a piperazine ring. In addition, if A₁ is a radical chosen from linear and branched, saturated and unsaturated alkylene and hydroxyalkylene radicals, B₁ can also denote a group (CH₂)ₙ—CO-D-OC—(CH₂)ₙ—, wherein n ranges from 1 to 100, such as from 1 to 50,D is chosen from:

a) a glycol residue of formula: —O-Z-O—, wherein Z is chosen from linear and branched hydrocarbon-based radicals and a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y, which may be identical or different, are each an integer ranging from 1 to 4, representing a defined and unique degree of polymerization or any number ranging from 1 to 4 representing an average degree of polymerization;

b)a bis-secondary diamine residue such as a piperazine derivative;

c)a bis-primary diamine residue of formula: —NH—Y—NH—, where Y is chosen from linear and branched hydrocarbon radicals and the divalent radical —CH₂—CH₂—S—S—CH₂—CH₂—; and

d)a ureylene group of formula: —NH—CO—NH—.

In one embodiment, X⁻ is an anion such as chloride or bromide.

These polymers may have a number-average molecular mass ranging from 1000 to 100,000.

These polymers are described, for example, in French Patent Nos. 2 320 330, 2 270 846, 2 316 271, 2 336 434 and 2 413 907 and U.S. Pat. Nos. 2,273,780, 2 375 853, 2 388 614, 2 454 547, 3 206 462, 2 261 002, 2 271 378, 3 874 870, 4 001 432, 3 929 990, 3 966 904, 4 005 193, 4 025 617, 4 025 627, 4 025 653,4 026 945 and 4 027 020.

Further, according to the present invention, polymers can comprise repeating units corresponding to the formula (a):

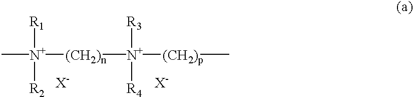

in which R₁, R₂, R₃ and R₄, which may be identical or different, are chosen from alkyl and hydroxyalkyl radicals comprising from 1 to 4 carbon atoms approximately, n and p, which may be identical or different, are integers ranging from 2 to 20, and X⁻ is an anion chosen from anions derived from inorganic and organic acids.

One compound of formula (a), for example, is the one for which R₁, R₂, R₃ and R₄ are each a methyl radical and n=3, p=6 and X=Cl, which is known as Hexadimethrine chloride according to the INCl (CTFA) nomenclature.

(9)polyquaternary ammonium polymers comprising repeating units of formula (III):

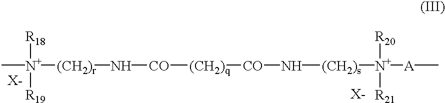

in which:

R₁₈, R₁₉, R₂₀ and R₂₁, which may be identical or different, are chosen from a hydrogen atom and methyl, ethyl, propyl, β-hydroxyethyl, β-hydroxypropyl and —CH₂CH₂(OCH₂CH₂)ₚOH radicals,

wherein p is equal to 0 or to an integer ranging from 1 to 6, with the proviso that R₁₈, R₁₉, R₂₀ and R₂₁ do not simultaneously represent a hydrogen atom,

r and s, which may be identical or different, are each an integer ranging from 1 to 6,

q is equal to 0 or to an integer ranging from 1 to 34,

X⁻ is an anion such as a halide,

A is chosen from divalent radicals, such as —CH₂—CH₂—O—CH₂—CH₂—. Such polymers are described, for example, in patent application EP-A-1 22 324.

Among these polymers, mention may be made, for example, of “Mirapol® A 15”, “Mirapol® AD1”, “Mirapol® AZ1” and “Mirapol® 175” sold by the company Miranol.

(10) quaternary polymers of vinylpyrrolidone and of vinylimidazole, such as, the products sold under the names Luviquat® FC 905, FC 550 and FC 370 by the company BASF.

(11)Polyamines.

(12)Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide.

Other cationic polymers, which can be used in the context of the invention, are chosen from cationic proteins and cationic protein hydrolysates, polyalkyleneimines, such as polyethyleneimines, polymers comprising units chosen from vinylpyridine units and vinylpyridinium units, condensates of polyamines and of epichlorohydrin, quaternary polyureylenes and chitin derivatives.

Among all the cationic polymers that may be used in the context of the present invention, non-limiting examples include cationic cyclopolymers, such as the dimethyldiallylammonium chloride homopolymers or copolymers sold under the names “Merquat 100”, “Merquat 550” and “Merquat S” by the company Nalco, quaternary polymers of vinylpyrrolidone and of vinylimidazole, crosslinked homopolymers or copolymers of methacryloyloxy(C₁-C₄)alkyltri(C₁-C₄)alkylammonium salts, and mixtures thereof.

The amphoteric polymers, which may be used in accordance with the present invention, may be chosen from polymers comprising units K and M randomly distributed in the polymer chain, in which K is a unit derived from a monomer comprising at least one basic nitrogen atom and M is a unit derived from an acidic monomer comprising at least one carboxylic or sulphonic group, or K and M may be chosen from groups derived from zwitterionic carboxybetaine or sulphobetaine monomers.

K and M may also be chosen from a cationic polymer chain comprising at least one group chosen from primary, secondary, tertiary and quaternary amine groups, in which at least one of the amine groups bears a carboxylic or sulphonic group linked via a hydrocarbon-based radical, or K and M can form part of a chain of a polymer comprising an α,β-dicarboxylic ethylene unit in which one of the carboxylic groups has been made to react with a polyamine comprising at least one amine chosen from primary and secondary amine groups.

The amphoteric polymers corresponding to the above definition, for example, are chosen from the following polymers:

(1)polymers resulting from the copolymerization of at least one monomer derived from a vinyl compound bearing a carboxylic group such as acrylic acid, methacrylic acid, maleic acid, α-chloroacrylic acid, and at least one basic monomer derived from a substituted vinyl compound comprising at least one basic atom, such as dialkylaminoalkyl methacrylate and dialkylaminoalkyl acrylate, dialkylaminoalkylmethacrylamide and dialkylaminoalkylacrylamide. Such compounds are described, for example, in U.S. Pat. No. 3,836,537.

Mention may also be made of the sodium acrylate/acrylamidopropyltrimethylammonium chloride copolymer.

The vinyl compound may also be a dialkyldiallylammonium salt such as dimethyldiallylammonium chloride. The copolymers of acrylic acid and of the latter monomer are sold under the name Merquat 280 by the company Nalco.

(2)polymers comprising units derived from:

a)at least one monomer chosen from acrylamides and methacrylamides substituted on the nitrogen with an alkyl radical,

b)at least one acidic comonomer comprising at least one reactive carboxylic group, and

c)at least one basic comonomer such as esters comprising substituents chosen from primary, secondary, tertiary and quaternary amine substituents of acrylic and methacrylic acids and the product of quaternization of dimethylaminoethyl methacrylate with dimethyl or diethyl sulphate.

In one embodiment, the N-substituted acrylamides or methacrylamides according to the invention are, for example, groups in which the alkyl radicals comprise from 2 to 12 carbon atoms, such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acids, methacrylic acids, crotonic acids, itaconic acids, maleic acids and fumaric acids and alkyl monoesters, comprising 1 to 4 carbon atoms, of maleic or fumaric acids or anhydrides.

The basic comonomers are chosen, for example, from aminoethyl, butylaminoethyl, N,N′-dimethylaminoethyl and N-tert-butylaminoethyl methacrylates.

The copolymers having the CTFA (4th edition, 1991) name octylacrylamide/acrylates/butylaminoethyl methacrylate copolymer, for example, also can be used.

(3)crosslinked and alkylated polyamino amides partially or totally derived from polyamino amides of general formula:

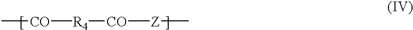

in which R₄ is chosen from a divalent radical derived from saturated dicarboxylic acid, mono- and dicarboxylic aliphatic acids comprising an ethylenic double bond, an ester of a lower alkanol, comprising from 1 to 6 carbon atoms, of these acids and a radical derived from the addition of any one of the acids to amines chosen from bis(primary) and bis(secondary) amines, and Z is chosen from bis(primary), mono- and bis(secondary) polyalkylene-polyamine radicals and, for example, Z represents:

a)in proportions of from 60 to 100 mol %, the radical

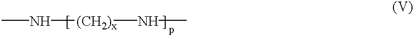

wherein x=2 and p=2 or 3,or x=3 and p=2,

this radical being derived from a compound chosen from diethylenetriamine, triethylenetetraamine and dipropylenetriamine;

b)in proportions of from 0 to 40 mol %, the radical (V) above in which x=2 and p=1 and which is derived from a compound chosen from ethylenediamine and piperazine:

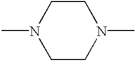

c)in proportions of from 0 to 20 mol %, the —NH—(CH₂)₆—NH— radical, which is derived from hexamethylenediamine, these polyamino amides can be crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

In one embodiment, the saturated carboxylic acids are, for example, chosen from acids comprising 6 to 10 carbon atoms, such as adipic acid, 2,2,4-trimethyladipic acid and 2,4,4-trimethyladipic acid, terephthalic acid and acids comprising an ethylenic double bond such as acrylic acid, methacrylic acid and itaconic acid.

The alkane sultones used in the alkylation are chosen, for example, from propane sultone and butane sultone, and the salts of the alkylating agents can be chosen, for example, from sodium and potassium salts.

(4)polymers comprising zwitterionic units of formula:

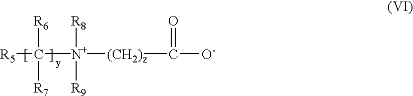

in which R₅ is chosen from polymerizable unsaturated groups, such as acrylate, methacrylate, acrylamide and methacrylamide groups,

y and z, which may be identical or different, are chosen from integers ranging from 1 to 3,

R₆ and R₇, which may be identical or different, are chosen from a hydrogen atom, methyl, ethyl and propyl groups,

R₈ and R₉, which may be identical or different, are chosen from a hydrogen atom and alkyl radicals such that the sum of the carbon atoms in R₈ and R₉ does not exceed 10.

The polymers comprising such units can also comprise units derived from non-zwitterionic monomers such as monomers chosen from dimethyl and diethylaminoethyl acrylates and methacrylates, alkyl acrylates, methacrylates, acrylamides, methacrylamides, and vinyl acetate.

By way of example, mention may be made of the copolymer of butyl methacrylate/dimethylcarboxymethylammonioethyl methacrylate.

(5)polymers derived from chitosan comprising monomer units corresponding to formulae (VII), (VIII) and (IX) below:

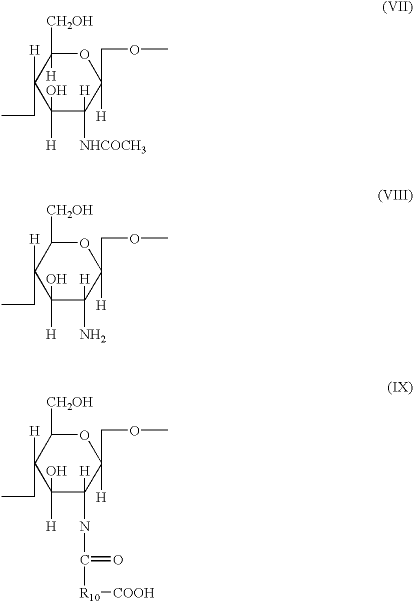

the unit (VII) being present in proportions ranging from 0 to 30%, the unit (VIII) in proportions ranging from 5% to 50% and the unit (IX) in proportions ranging from 30% to 90%, and wherein in the unit (IX), R₁₀ is a radical of formula:

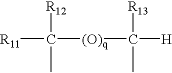

wherein q is equal to 0 or 1;

if q=0, R₁₁, R₁₂ and R₁₃, which may be identical or different, are each chosen from a hydrogen atom, methyl, hydroxyl, acetoxy and amino residues, monoalkylamine residues and dialkylamine residues, which are optionally interrupted by at least one nitrogen atom and/or optionally substituted with at least one group chosen from amine, hydroxyl, carboxyl, alkylthio and sulphonic groups, and alkylthio residues in which the alkyl group bears an amino residue, at least one of the radicals R₁₁, R₁₂ and R₁₃ being, in this case, a hydrogen atom;

or, if q=1, R₁₁, R₁₂ and R₁₃, which may be identical or different, are each chosen from a hydrogen atom, and the salts formed by these compounds with bases or acids.

(6)polymers derived from the N-carboxyalkylation of chitosan, such as N-carboxymethylchitosan and N-carboxybutylchitosan.

(7)polymers corresponding to the general formula (X) as described, for example, in French Patent No. 1 400 366:

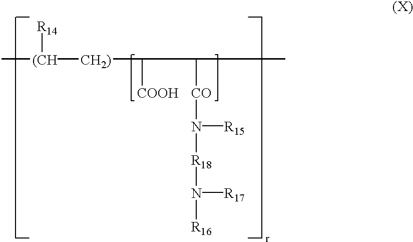

in which R₁₄ is chosen from a hydrogen atom, CH₃O, CH₃CH₂O and phenyl radicals,

R₁₅ is chosen from a hydrogen atom and lower alkyl radicals such as methyl and ethyl,

R₁₆ is chosen from a hydrogen atom and lower alkyl radicals such as methyl and ethyl,

R₁₇ is chosen from lower alkyl radicals such as methyl and ethyl, radicals corresponding to the formula: —R₁₈—N(R₁₆)₂, wherein R₁₈ is chosen from —CH₂—CH₂—, —CH₂—CH₂—CH₂— and —CH₂—CH(CH₃)— groups, and R₁₆ is chosen from a hydrogen atom and lower alkyl radicals such as methyl and ethyl,

and the higher homologues of these radicals comprising up to 6 carbon atoms

r is chosen such that the number-average molecular weight of said polymer ranges from 500 to 6,000,000, such as from 1,000 to 1,000,000.

(8)amphoteric polymers of the type -D-X-D-X— chosen from:

a) polymers obtained by the action of chloroacetic acid or sodium chloroacetate on compounds comprising at least one unit of formula:

-D-X-D-X-D- (XI)

wherein D is a radical

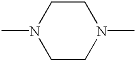

and X is chosen from the symbols E and E′, wherein E and E′, which may be identical or different, are chosen from divalent alkylene radicals comprising at least one chain chosen from straight and branched chains comprising up to 7 carbon atoms in the main chain, wherein said divalent alkylene radicals are optionally substituted with at least one hydroxyl group. E or E′ can additionally comprise at least one atom chosen from oxygen, nitrogen and sulphur atoms, and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine, alkenylamine, hydroxyl, benzylamine, amine oxide, quaternary ammonium, amide, imide, alcohol, ester and urethane groups;

b)polymers of formula:

-D-X-D-X— (XI)

wherein D is a radical

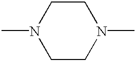

and X is chosen from the symbols E and E′ and wherein at least one X is chosen from E′; E having the meaning given above and E′ being chosen from divalent alkylene radicals comprising at least one chain chosen from straight and branched chains comprising up to 7 carbon atoms in the main chain, wherein said divalent alkylene radicals are optionally substituted with at least one hydroxyl radical. E′ can also comprise at least one nitrogen atom substituted with an alkyl chain, which is optionally interrupted by an oxygen atom, wherein said alkyl chain comprises at least one functional group chosen from carboxyl functional groups and hydroxyl functional groups, and wherein the alkyl chain is betainized by reaction with a reactant chosen from chloroacetic acid and sodium chloroacetate.

(9)(C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

The amphoteric polymers according to the invention are, in certain embodiments, those of family (1), such as dimethyldiallyammonium salt/acrylic acid copolymers.

According to the invention, the at least one polymer chosen from cationic and amphoteric polymers may be present in an amount ranging, for example, from 0.001% to 20% by weight, such as from 0.01% to 10% by weight and further such as from 0.1% to 5% by weight relative to the total weight of the final composition.

According to one embodiment of the present invention, the compositions further comprise at least one additional silicone other than silicones comprising at least one dimethylsilanol group or another agent that is beneficial for keratin materials, such as the hair, for example, esters of C₁-C₃₀ carboxylic acids and of C₁-C₃₀ mono- or polyhydroxylated alcohols, plant, animal, mineral or synthetic oils, waxes, ceramides and pseudoceramides.

According to the invention, all the silicones and beneficial agents may also be used in the form of emulsions, nanoemulsions or microemulsions.

The at least one additional silicone in accordance with the invention includes, for example:

-non-volatile silicones chosen from the family of polyalkylsiloxanes comprising trimethylsilyl end groups, such as oils with a viscosity ranging from 0.2 to 2.5 m²/s at 25° C. such as the oils of the DC200 series from Dow Corning, for example, the product of viscosity 60 000 Cst, of the Silbione 70047 and 47 series and further for example, the oil 70 047 V 500 000 sold by the company Rhodia Chimie, and aminosilicones such as amo-dimethicones and trimethylsilylamodimethicones.

According to the invention, the at least one additional silicone or the other additional beneficial agents can be present in an amount ranging from 0.001% to 20% by weight, such as from 0.01% to 10% by weight and further such as from 0.1% to 5% by weight relative to the total weight of the composition.

The compositions of the invention can also comprise at least one surfactant, which is generally present in an amount ranging from 0.01% to 50% by weight, such as from 0.1% to 40% by weight and further such as from 0.5% to 30% by weight relative to the total weight of the composition.

The at least one surfactant may be chosen from anionic, amphoteric, nonionic and cationic surfactants.

The at least one surfactant that is suitable for carrying out the present invention is, for example, chosen from:

(i) Anionic surfactants:

As examples of anionic surfactants, which can be used, alone or as mixtures, in the context of the present invention, mention may be made, for example, of salts (such as alkaline salts, for example, sodium salts, ammonium salts, amine salts, amino alcohol salts and magnesium salts) of the following compounds: alkyl sulphates, alkyl ether sulphates, alkylamidoether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates and N-acyltaurates, the alkyl or acyl radical of all of these various compounds, for example, comprising from 8 to 24 carbon atoms, and the aryl radical is chosen, for example, from phenyl and benzyl groups. Among the anionic surfactants, which can also be used, mention may also be made of fatty acid salts such as the salts of oleic, ricinoleic, palmitic and stearic acids, coconut oil acid or hydrogenated coconut oil acid; acyl lactylates in which the acyl radical comprises from 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, such as those comprising from 2 to 50 ethylene oxide groups, and mixtures thereof. Among the anionic surfactants, for example, alkyl sulphate salts and alkyl ether sulphate salts and mixtures thereof can be used.

(ii) Nonionic surfactants:

The nonionic surfactants are compounds that are well known (see, for example, in this respect “Handbook of Surfactants” by M. R. Porter, published by Blackie & Son (Glasgow and London), 1991, pp. 1 16-178). They can be chosen, for example, from polyethoxylated, polypropoxylated and polyglycerolated fatty acids, alkylphenols, a-diols and alcohols comprising a fatty chain comprising, for example, from 8 to 18 carbon atoms, it being possible for the number of ethylene oxide or propylene oxide groups to range, for example, from 2 to 50 and for the number of glycerol groups to range, for example, from 2 to 30. Mention may also be made of copolymers of ethylene oxide and of propylene oxide, condensates of ethylene oxide and of propylene oxide with fatty alcohols; polyethoxylated fatty amides, for example, those comprising from 2 to 30 mol of ethylene oxide, polyglycerolated fatty amides comprising on average from 1 to 5, and such as from 1.5 to 4, glycerol groups; polyethoxylated fatty amines, for example, those comprising from 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It will be noted that the alkylpolyglycosides constitute nonionic surfactants that can be used in the context of the present invention.

(iii) Amphoteric surfactants:

The amphoteric surfactantscan be chosen, for example, from aliphatic secondary and tertiary amine derivatives in which the aliphatic radical is chosen from linear and branched chains comprising from 8 to 22 carbon atoms and comprising at least one water-soluble anionic group (for example carboxylate, sulphonate, sulphate, phosphate or phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the (C₈-C₂₀)alkylamido(C₁-C₆)alkylbetaines that may be mentioned is the cocoamidopropylbetaine sold, for example, by Goldschmidt under the name Tegobetaine F50.

Among the amine derivatives, mention may be made of the products sold under the name Miranol, as described, for example, in U.S. Pat. Nos. 2,528,378 and 2 781 354 and having the structures of:

R₂—CONHCH₂CH₂—N⁺(R₃)(R₄)(CH₂COO⁻)(2)

in which: R₂ is chosen from alkyl radicals derived from an acid R₂—COOH present in hydrolyzed coconut oil, and heptyl, nonyl and undecyl radicals, R₃ is a β-hydroxyethyl group and R₄ is a carboxymethyl group;

and of

R₅—CONHCH₂CH₂—N(B)(C)(3)

wherein B represents —CH₂CH₂OX′, C represents —(CH₂)₂-Y′, with z=1 or 2,

X′ is chosen from the —CH₂CH₂—COOH group and a hydrogen atom,

Y′ is chosen from the —COOH and the —CH₂—CHOH—SO₃H radicals,

R₅ is chosen from alkyl radicals of an acid R₅—COOH present in coconut oil orin hydrolyzed linseed oil, alkyl radicals, such as C₇, C₉, C₁₁, and C₁₃ alkyl radicals, a C₁₇ alkyl radical and its iso form, and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylampho-dipropionate, disodium capryloamphodipropionate, lauroamphodipropionic acid, and cocoamphodipropionic acid.

By way of example, mention may be made of the cocoamphodiacetate sold under the trade name Miranol C2M concentrated by the company Rhodia Chimie.

(iv) The cationic surfactants may be chosen from:

A)the quaternary ammonium salts of general formula (XIII) below:

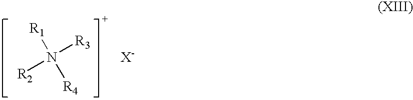

in which X⁻ is an anion chosen from halides (chloride, bromide and iodide), (C₂-C₆)alkyl sulphates, such as methyl sulphate, phosphates, alkyl and alkylaryl sulphonates, and anions derived from organic acids, such as acetate and lactate, and

a)the radicals R₁ to R₃, which may be identical or different, are chosen from linear and branched aliphatic radicals comprising from 1 to 4 carbon atoms, and aromatic radicals such as aryl and alkylaryl. The aliphatic radicals can comprise at least one hetero atom such as oxygen, nitrogen, sulphur and halogen. The aliphatic radicals are chosen, for example, from alkyl, alkoxy and alkylamide radicals,

R₄ is chosen from linear and branched alkyl radicals comprising from 16 to 30 carbon atoms.

The cationic surfactant is, for example, a behenyltrimethylammonium salt (for example chloride).

b)the radicals R₁ and R₂, which may be identical or different, are chosen from linear and branched aliphatic radicals comprising from 1 to 4 carbon atoms, and aromatic radicals such as aryl and alkylaryl. The aliphatic radicals can comprise at least one hetero atom such as oxygen, nitrogen, sulphur and halogen. The aliphatic radicals are chosen, for example, from alkyl, alkoxy, alkylamide and hydroxyalkyl radicals comprising from about 1 to 4 carbon atoms;

R₃ and R₄, which may be identical or different, are chosen from linear and branched alkyl radicals comprising from 12 to 30 carbon atoms, the alkyl radicals may comprise at least one ester or amide function.

R₃ and R₄ are chosen, for example, from (C₁₂-C₂₂)alkylamido(C₂-C₆)alkyl and (C₁₂-C₂₂)alkylacetate radicals.

The cationic surfactant is, for example, a stearamidopropyldimethyl(myristyl acetate)ammonium salt (for example chloride);

B)—the quaternary ammonium salts of imidazolinium, such as that of formula (XIV) below:

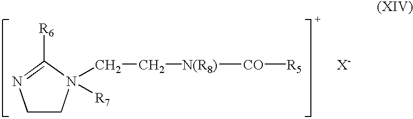

in which R₅ is chosen from alkenyl and alkyl radicals comprising from 8 to 30 carbon atoms, for example fatty acid derivatives of tallow,

R₆ is chosen from a hydrogen atom, C₁-C₄ alkyl radicals, and alkenyl and alkyl radicals comprising from 8 to 30 carbon atoms,

R₇ is chosen from C₁-C₄ alkyl radicals,

R₈ is chosen from a hydrogen atom and C₁-C₄ alkyl radicals, and

X⁻ is an anion chosen from halides, phosphates, acetates, lactates, alkyl sulphates, alkyl sulphonates and alkylaryl sulphonates.

In one embodiment, R₅ and R₆ are, for example, a mixture of radicals chosen from alkenyl and alkyl radicals comprising from 12 to 21 carbon atoms, such as fatty acid derivatives of tallow, R₇ denotes methyl and R₈ is hydrogen. Such a product is, for example, Quaternium-27 (CTFA 1997) or Quaternium-83 (CTFA 1997), which are sold under the names “Rewoquat” W75, W90, W75PG and W75HPG by the company Witco,

C)—the diquaternary ammonium salts of formula (XV):

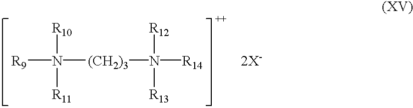

in which R₉ is chosen from aliphatic radicals comprising from 16 to 30 carbon atoms,

R₁₀, R₁₁, R₁₂, R₁₃ and R₁₄, which may be identical or different, are chosen from a hydrogen atom and alkyl radicals comprising from 1 to 4 carbon atoms, and

X⁻ is an anion chosen from halides, acetates, phosphates, nitrates and methyl sulphates.

Such diquaternary ammonium salts, for example, include propanetallowdiammonium dichloride; and

D)—the quaternary ammonium salts comprising at least one ester function, of formula (XVI) below:

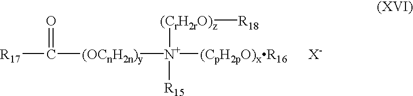

in which:

R₁₅ is chosen from C₁-C₆ alkyl radicals and C₁-C₆ hydroxyalkyl and dihydroxyalkyl radicals;

R₁₆ is chosen from:

a radical

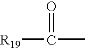

linear and branched, saturated and unsaturated C₁-C₂₂ hydrocarbon-based radicals R₂₀, and

a hydrogen atom,

R₁₈ is chosen from:

a radical

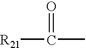

linear and branched, saturated and unsaturated C₁-C₆ hydrocarbon-based radicals R₂₂, and

a hydrogen atom,

R₁₇, R₁₉ and R₂₁, which may be identical or different, are chosen from linear and branched, saturated and unsaturated C₇-C₂₁ hydrocarbon-based radicals;

n, p and r, which may be identical or different, are chosen from integers ranging from 2 to 6;

y is chosen from integers ranging from 1 to 10;

x and z, which may be identical or different, are chosen from integers ranging from 0 to 10;

X⁻ is an anion chosen from simple and complex, organic and inorganic anions;

with the proviso that the sum x+y+z is from 1 to 15, that when x is 0, then R₁₆ is R₂₀ and that when z is 0, then R₁₈ is R₂₂.

In one embodiment, the ammonium salts of formula (XVI) can be used, in which:

R₁₅ is chosen from methyl and ethyl radicals,

x and y are equal to 1;

z is equal to 0 or 1;

n, p and r are equal to 2;

R₁₆ is chosen from:

a radical

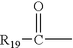

methyl, ethyl and C₁₄-C₂₂ hydrocarbon-based radicals, and

a hydrogen atom;

R₁₇, R₁₉ and R₂₁, which may be identical or different, are chosen from linear and branched, saturated and unsaturated C₇-C₂₁ hydrocarbon-based radicals;

R₁₈ is chosen from:

a radical

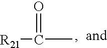

a hydrogen atom.

Such compounds are sold, for example, under the names Dehyquart by the company Cognis, Stepanquat by the company Stepan, Noxamium by the company Ceca, and Rewoquat WE 18 by the company Rewo-Witco.

Among the quaternary ammonium salts, examples are behenyltrimethylammonium chloride and stearamidopropylmethyl(myristyl acetate)ammonium chloride, sold under the name “Ceraphyl 70” by the company Van Dyk, and Quaternium-27 or Quaternium-83 sold by the company Witco.

In the compositions in accordance with the invention, mixtures of surfactants and, such as mixtures of anionic surfactants, mixtures of anionic surfactants and of at least one surfactant chosen from amphoteric, cationic and nonionic surfactants, and mixtures of cationic surfactants with at least one surfactant chosen from nonionic and amphoteric surfactants may be used. One mixture, for example, is a mixture comprising at least one anionic surfactant and at least one amphoteric surfactant.

The composition of the invention may also comprise at least one additive chosen from thickeners, fragrances, nacreous agents, preserving agents, silicone and non-silicone sunscreens, vitamins, provitamins, anionic and nonionic polymers, non-cationic proteins, non-cationic protein hydrolysates, 18-methyleicosanoic acid, hydroxy acids, vitamins, provitamins such as panthenol, and any other additive conventionally used in cosmetics that does not affect the properties of the compositions according to the invention.

The compositions in accordance with the invention may also comprise up to 5% of nacreous or opacifying agents that are well known, such as sodium or magnesium palmitate, sodium or magnesium stearate or hydroxystearate, fatty-chain acyl derivatives such as ethylene glycol or polyethylene glycol monostearates or distearates, fatty-chain ethers such as distearyl ether or 1-(hexadecyloxy)-2-octadecanol, and fatty alcohols, such as stearyl alcohol, cetyl alcohol or behenyl alcohol, and mixtures thereof.

These additives are optionally present in the composition according to the invention in proportions that can range from 0.001% to 20% by weight relative to the total weight of the composition. The precise amount of each additive is readily determined by a person skilled in the art on the basis of its nature and its function.

The physiologically and cosmetically acceptable medium may consist solely of water or of a mixture of water and a cosmetically acceptable solvent such as a C₁-C₄ lower alcohol, for instance ethanol, isopropanol, tert-butanol or n-butanol; alkylene glycols, for instance propylene glycol, and glycol ethers.

For example, the composition comprises from 50% to 95% by weight of water relative to the total weight of the composition, and further, for example, from 65% to 90% by weight of water relative to the total weight of the composition.

The compositions according to the invention can have a final pH generally ranging from 3 to 10. For example, this pH is ranging from 4 to 8. Adjusting the pH to the desired value may be performed conventionally by adding a base (organic or mineral base) to the composition, for example aqueous ammonia or a primary, secondary or tertiary (poly)amine, for instance monoethanolamine, diethanolamine, triethanolamine, isopropanolamine or 1,3-propanediamine, or by adding a mineral or organic acid, such as a carboxylic acid, for example, citric acid.

The compositions in accordance with the invention may be used, for example, for washing or treating keratin materials such as the hair, the skin, the eyelashes, the eyebrows, the nails, the lips or the scalp.

The compositions according to the invention may be detergent compositions such as shampoos, shower gels and bubble baths. In this embodiment of the invention, the compositions comprise at least one washing base, which is generally aqueous.

The at least one washing base comprises at least one detergent surfactant. The at least one surfactant may be chosen, without discrimination, alone or as mixtures, from the anionic, amphoteric, nonionic and cationic surfactants as defined above.

In the compositions in accordance with the invention, at least one anionic surfactant or mixtures of at least one anionic surfactant and of at least one surfactant chosen from amphoteric surfactants and nonionic surfactants can be, for example, used.

In one embodiment, a mixture, for example, is a mixture comprising at least one anionic surfactant and at least one amphoteric surfactant.

For example, one can use an anionic surfactant chosen from sodium, triethanolamine and ammonium (C₁₂-C₁₄)alkyl sulphates, sodium, triethanolamine and ammonium (C₁₂-C₁₄)alkyl ether sulphates oxyethylenated with 2.2 mol of ethylene oxide, sodium cocoyl isethionate and sodium α-(C₁₄-C₁₆)olefin sulphonate, and mixtures thereof with:

either an amphoteric surfactant such as the amine derivatives known as disodium cocoamphodipropionate and sodium cocoamphopropionate sold, for example, by the company Rhodia Chimie under the trade name “Miranol C2M CONC” as an aqueous solution comprising 38% active material, or under the name Miranol C32; or -an amphoteric surfactant of zwitterionic type, such as alkylbetaines and alkylamidobetaines and, for example, the cocobetaine sold under the name “Dehyton AB 30” as an aqueous solution comprising 32% AM by the company Cognis, or the cocoamidopropylbetaine sold, for example, by Goldschmidt under the name Tegobetaine F50.

The quantity and quality of the washing base are those that are sufficient to be able to give the final composition satisfactory foaming power and/or detergent power.

These detergent compositions are, for example, foaming and the foaming power of the compositions according to the invention, characterized by a foam height, is generally greater than 75 mm and, for example, greater than 100 mm, measured according to the modified Ross-Miles method (NF T 73-404/IS696).

The modifications to the method are the following:

The measurement is performed at a temperature of 22° C. with osmosed water. The concentration of the solution is 2 g/l. The height of the drop is 1 m. The amount of composition that is dropped is 200 ml. These 200 ml of composition fall into a measuring cylinder 50 mm in diameter and containing 50 ml of the test composition. The measurement is taken 5 minutes after stopping the flow of the composition.

Thus, according to the invention, the washing base can be in an amount ranging from 3% to 50% by weight, such as from 6% to 35% by weight and further such as from 8% to 25% by weight relative to the total weight of the final composition.

Another aspect of the invention is a process for treating a keratin material such as the skin or the hair, characterized in that the process comprises applying to the keratin material a cosmetic composition as defined above, and then optionally rinsing it out with water.

Thus, this process according to the invention can allow the maintenance of the hairstyle and the treatment, care, washing or removal of makeup of the skin, the hair or any other keratin material.

The compositions of the invention may also be in the form of rinse-out or leave-in conditioners, permanent-waving, hair-straightening, dyeing or bleaching compositions, or in the form of rinse-out compositions to be applied before or after dyeing, bleaching, permanent-waving or straightening the hair or between the two steps of a permanent-waving or hair-straightening operation.

When the composition is in the form of a conditioner, such as a rinse-out conditioner, it, for example, comprises at least one cationic surfactant, and its concentration is generally from 0.1% to 10% by weight, and such as from 0.5% to 5% by weight relative to the total weight of the composition.

The compositions of the invention may also be in the form of washing compositions for the skin, such as in the form of bath or shower solutions or gels or makeup-removing products.

The compositions according to the invention may also be in the form of aqueous or aqueous-alcoholic lotions for skincare and/or haircare.

The cosmetic compositions according to the invention may be in the form of a gel, a milk, a cream, an emulsion, a thickened lotion or a mousse and may be used for the skin, the nails, the eyelashes, the lips and, for example, the hair.

The compositions may be packaged in various forms, such as in vaporizers, pump-dispenser bottles or in aerosol containers to allow an application of the composition in vaporized form or in the form of a mousse. Such packaging forms are indicated, for example, when it is desired to obtain a spray, a lacquer or a mousse for treating keratin materials, such as the hair.

Throughout the text hereinabove and hereinbelow, the percentages expressed are on a weight basis.

The invention will now be illustrated more fully with the aid of the examples that follow, which cannot be considered as limiting it to the embodiments described.

In the examples, AM means active material.

### EXAMPLE I

A shampoo in accordance with the invention, comprising the composition below, was prepared:

| Composition | Example 1 | |
|---|---|---|
| Sodium lauryl ether sulphate containing 2.2 mol of | 15 g | AM |
| ethylene oxide, as an aqueous solution containing 70% | | |
| AM | | |
| Cocoylamidopropylbetaine as an aqueous solution | 2.4 g | AM |
| containing 30% AM | | |
| Methacrylic acid/ethyl acrylate crosslinked copolymer as | 1.1 g | AM |
| an aqueous emulsion containing 30% AM, sold under | | |
| the name Carbopol Aqua SF1 by the company Noveon | | |
| Polydimethylsiloxane containing dimethylsilanol end | 2 g | |
| groups, sold as an aqueous 50% emulsion under the | | |
| name DC1784 by the company Dow Corning | | |
| Dimethyldiallylammonium chloride homopolymer, as an | 1.2 g | |
| aqueous solution containing 40% AM, sold under the | | |
| name Merquat 100 by the company Nalco | | |
| Behenyl alcohol | 1.5 g | |
| Distearyl ether | 1.5 g | |
| Preserving agents | q.s. | |
| Citric acid or sodium hydroxideq.s. | pH 5.5 | |
| Demineralized water q.s. | 100 g | |

The composition is stable at least one week at an ambient temperature (about 20-25° C.). Moistened hair is not laden and is easy to shape.

What is claimed is:

1. A cosmetic composition, comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

2. The composition according to claim 1, wherein in said at least one copolymer, the methacrylic acid residue is in an amount ranging from 20% to 80% by weight relative to the total weight of the copolymer.

3. The composition according to claim 2, wherein in said at least one copolymer, the methacrylic acid residue is in an amount ranging from 25% to 70% by weight relative to the total weight of the copolymer.

4. The composition according to claim 3, wherein in said at least one copolymer, the methacrylic acid residue is in an amount ranging from 35% to 60% by weight relative to the total weight of the copolymer.

5. The composition according to claim 1, wherein in said at least one copolymer, the alkyl acrylate residue is in an amount ranging from 15% to 80% by weight relative to the total weight of the copolymer.

6. The composition according to claim 5, wherein in said at least one copolymer, the alkyl acrylate residue is in an amount ranging from 25% to 75% by weight relative to the total weight of the copolymer.

7. The composition according to claim 6, wherein in said at least one copolymer, the alkyl acrylate residue is in an amount ranging from 40% to 65% by weight relative to the total weight of the copolymer.

8. The composition according to claim 1, wherein in said at least one copolymer, the alkyl acrylate residue is chosen from methyl acrylate, ethyl acrylate, and butyl acrylate residues.

9. The composition according to claim 8, wherein the alkyl acrylate residue is ethyl acrylate residue.

10. The composition according to claim 1, wherein the at least one copolymer of methacrylic acid and of a C₁-C₄ alkyl acryl ate is crosslinked.

11. The composition according to claim 1, wherein the at least one copolymer of methacrylic acid and of a C₁-C₄ alkyl acrylate is partially or totally crosslinked with at least one crosslinking agent.

12. The composition according to claim 11, wherein the at least one crosslinking agent is an ethylenically polyunsaturated crosslinking agent.

13. The composition according to claim 11, wherein the content of the at least one crosslinking agent ranges from 0.01% to 5% by weight relative to the total weight of the copolymer.

14. The composition according to claim 13, wherein the content of the at least one crosslinking agent ranges from 0.03% to 3% by weight relative to the total weight of the copolymer.

15. The composition according to claim 14, wherein the content of the at least one crosslinking agent ranges from 0.05% to 1% by weight relative to the total weight of the copolymer.

16. The composition according to claim 1, wherein the at least one copolymer of methacrylic acid and of a C₁-C₄ alkyl acrylate is in the form of a dispersion of particles in water.

17. The composition according to claim 16, wherein the number-average size of the particles of the copolymer in the dispersion ranges from 10 to 500 nm.

18. The composition according to claim 17, wherein the number-average size of the particles of the copolymer in the dispersion ranges from 20 to 200 nm.

19. The composition according to claim 18, wherein the number-average size of the particles of the copolymer in the dispersion ranges from 50 to 150 nm.

20. The composition according to claim 1, wherein the at least one copolymer of methacrylic acid and of a C₁-C₄ alkyl acrylate is the methacrylic acid/ethyl acrylate crosslinked copolymer in the form of an aqueous dispersion at 30% by weight.

21. The composition according to claim 1, wherein said cationic polymers are chosen from those comprising at least one group chosen from primary, secondary, tertiary and quaternary amine groups that may either form part of the main polymer chain, or be borne by a side substituent that is directly attached to the main polymer chain.

22. The composition according to claim 1, wherein said cationic polymers are chosen from:
(1) homopolymers and copolymers derived from acrylic or methacrylic esters or amides and comprising at least one of the units of the following formulae: 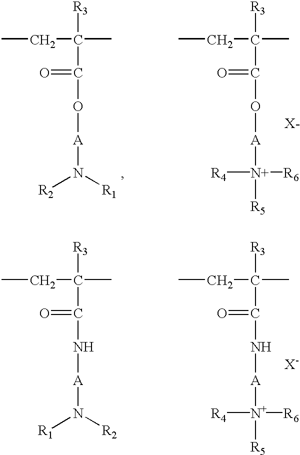
wherein:
R₃, which may be identical or different, is chosen from a hydrogen atom and a CH₃ radical;
A, which may be identical or different, is chosen from linear and branched alkyl groups of 1 to 6 carbon atoms, and hydroxyalkyl groups of 1 to 4 carbon atoms;
R₄, R₅ and R₆, which may be identical or different, are chosen from alkyl groups comprising from 1 to 18 carbon atoms and benzyl radicals;
R₁ and R₂, which may be identical or different, are chosen from a hydrogen atom and alkyl groups comprising from 1 to 6 carbon atoms;
X⁻ is an anion derived from an acid chosen from inorganic and organic acids;
(2) cationic polysaccharides;
(3) polymers comprising at least one piperazinyl unit and at least one radical chosen from divalent alkylene and hydroxyalkylene radicals comprising at least one chain chosen from straight and branched chains, optionally interrupted by at least one entity chosen from oxygen, sulphur and nitrogen atoms, and aromatic and heterocyclic rings, and oxidation and/or quaternization products of the polymers;
(4) water-soluble polyamino amides prepared by polycondensation of an acidic compound with a polyamine; optionally the polyamino amides being crosslinked with a crosslinking agent chosen from an epihalohydrin, a diepoxide, a dianhydride, an unsaturated dianhydride, a bis-unsaturated derivative, a bis-halohydrin, a bis-azetidinium, a bis-haloacyidiamine, a bis-alkyl halide and an oligomer resulting from the reaction of a difunctional compound which is reactive with a compound chosen from a bis-halohydrin, a bis-azetidinium, a bis-haloacyldiamine, a bis-alkyl halide, an epihalohydrin, a diepoxide and a bis-unsaturated derivative, wherein the crosslinking agent is in an amount ranging from 0.025 to 0.35 mol per amine group of the polyamino amides; further optionally, the polyamino amides being alkylated; even further optionally, if the polyamino amides comprise at least one tertiary amine function, the polyamino amides can be quaternized;
(5) polyamino amide derivatives resulting from the condensation of polyalkylene polyamines with polycarboxylic acids followed by alkylation with difunctional agents;
(6) polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 8 carbon atoms;
(7) cyclopolymers chosen from cyclopolymers of alkyldiallylamine and cyclopolymers of dialkyldiallylammonium;
(8) quaternary diammonium polymers comprising repeating units corresponding to the formula: 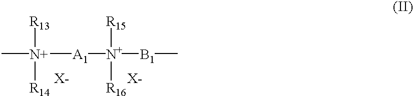
wherein:
R₁₃, R₁₄, R₁₅ and R₁₆, which may be identical or different, are chosen from aliphatic, alicyclic and arylaliphatic radicals comprising from 1 to 20 carbon atoms and lower hydroxyalkylaliphatic radicals, or R₁₃, R₁₄, R₁₅ and R₁₆, together or separately, constitute, with the nitrogen atoms to which they are attached, heterocycles optionally comprising a second hetero atom other than nitrogen, or R₁₃, R₁₄, R₁₅ and R₁₆ are chosen from linear and branched C₁-C₆ alkyl radicals substituted with a group chosen from nitrile, ester, acyl and amide groups and groups of —CO—O—R₁₇-D and —CO—NH—R₁₇-D, wherein R₁₇ is chosen from alkylene groups and D is chosen from quaternary ammonium groups;
A₁ and B₁, which may be identical or different, are chosen from linear and branched, saturated and unsaturated polymethylene groups comprising from 2 to 20 carbon atoms, and which may comprise, linked to or intercalated in the main chain, at least one entity chosen from aromatic rings, oxygen and sulphur atoms, and sulphoxide, sulphone, disulphide, amino, alkylamino, hydroxyl, quaternary ammonium, ureido, amide and ester groups; and
X⁻ is an anion chosen from anions derived from an acid chosen from inorganic and organic acids;
optionally A₁, R₁₃ and R₁₅ form, with the two nitrogen atoms to which they are attached, a piperazine ring; if A₁ is chosen from linear and branched, saturated and unsaturated alkylene and hydroxyalkylene radicals, B₁ can also be chosen from groups (CH₂)ₙ—CO-D-OC—(CH₂)ₙ—, wherein n ranges from 1 to 100, and D is chosen from:
a) glycol residues of formula: —O-Z-O—, wherein Z is chosen from linear and branched hydrocarbon-based radicals and groups corresponding to one of the following formulae:
—(CH₂—CH₂—O)ₓ—CH₂—CH₂— and
—[CH₂—CH(CH₃)—O]_{y}—CH₂—CH(CH₃)—
wherein x and y, which may be identical or different, are chosen from integers ranging from 1 to 4, representing a defined and unique degree of polymerization or any number ranging from 1 to 4 representing an average degree of polymerization;
b) bis-secondary diamine residues;
c) bis-primary diamine residues of formula: —NH—Y—NH—, wherein Y is chosen from linear and branched hydrocarbon radicals and the divalent radical
—CH₂—CH₂—S—S—CH₂—CH₂—; and
d) a ureylene group of formula: —NH—CO—NH—;
(9) polyquaternary ammonium polymers comprising units of formula (III): 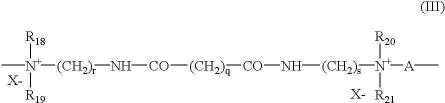
wherein:
R₁₈, R₁₉, R₂₀ and R₂₁, which may be identical or different, are chosen from a hydrogen atom and methyl, ethyl, propyl, β-hydroxyethyl, β-hydroxypropyl and —CH₂CH₂(OCH₂CH₂)ₚOH radicals,
wherein p is equal to 0 or is chosen from integers ranging from 1 to 6, with the proviso that R₁₈, R₁₉, R₂₀ and R₂₁ do not simultaneously represent a hydrogen atom,
r and s, which may be identical or different, are chosen from integers ranging from 1 to 6,
q is equal to 0 or chosen from integers ranging from 1 to 34,
X⁻ is a halogen anion, and
A is chosen from divalent radicals;
(10) quaternary polymers of vinylpyrrolidone and of vinylimidazole;
(11) crosslinked polymers of methacryloyloxy(C₁-C₄)alkyltri(C₁-C₄)alkylammonium salts; and
(12) polyalkyleneimines polymers comprising at least one unit chosen from vinylpyridine units and vinylpyridinium units, condensates of polyamines and of epichlorohydrin, quaternary polyureylenes and chitin derivatives.

23. The composition according to claim 22, wherein in (8)(b) the bis-secondary diamine residues are chosen from piperazine derivatives.

24. The composition according to claim 22, wherein in (12) the polyalkyleneimines are chosen from polyethyleneimines.

25. The composition according to claim 1, wherein said cationic polymers are chosen from cationic cyclopolymers, quaternary polymers of vinylpyrrolidone and of vinylimidazole, and crosslinked homopolymers and copolymers of methacryloyl-oxy(C₁-C₄)alkyltri(C₁-C₄)alkylammonium salts.

26. The composition according to claim 25, wherein the cationic cyclopolymers are chosen from diallyldimethylammonium chloride homopolymers and copolymers of diallyidimethylammonium chloride and of acrylamide.

27. The composition according to claim 1, wherein said amphoteric polymers are chosen from:
(1) polymers resulting from the copolymerization of at least one monomer derived from a vinyl compound bearing a carboxylic group and at least one basic monomer derived from a substituted vinyl compound comprising at least one basic atom, optionally the substituted vinyl compound is a dialkyldiallylammonium salt;
(2) polymers comprising at least one unit derived from:
a) at least one monomer chosen from acrylamides and methacrylamides substituted on the nitrogen with an alkyl radical,
b) at least one acidic comonomer comprising at least one reactive carboxylic group, and
c) at least one basic comonomer;
(3) crosslinked and alkylated polyamino amides partially or totally derived from polyamino amides of general formula: 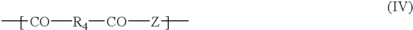
wherein R₄ is chosen from divalent radicals derived from a saturated dicarboxylic acid, mono- and dicarboxylic aliphatic acids comprising an ethylenic double bond, an ester of a lower alkanol, comprising 1 to 6 carbon atoms, of these acids and radicals derived from the addition of any one of the acids to an amine chosen from bis(primary) and bis(secondary) amines, and Z is chosen from bis(primary), mono- and bis(secondary) polyalkylene-polyamine radicals;
the polyamino amides being crosslinked by addition of a difunctional crosslinking agent chosen from epihalohydrins, diepoxides, dianhydrides and bis-unsaturated derivatives, using from 0.025 to 0.35 mol of the crosslinking agent per amine group of the polyamino amides and alkylated by the action of a reactant chosen from acrylic acids, chloroacetic acids and alkane sultones, and salts thereof;
(4) polymers comprising at least one zwitterionic unit of formula: 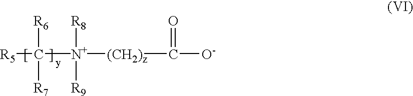
wherein R₅ is chosen from polymerizable unsaturated groups,
y and z, which may be identical or different, are chosen from integers ranging from 1 to 3,
R₆ and R₇, which may be identical or different, are chosen from a hydrogen atom, methyl, ethyl and propyl groups,
R₈ and R₉ are chosen from a hydrogen atom and alkyl radicals such that the sum of the carbon atoms in R₈ and R₉ does not exceed 10;
(5) polymers derived from chitosan comprising at least one monomer unit corresponding to formulae (VII), (VIII) and (IX) below: 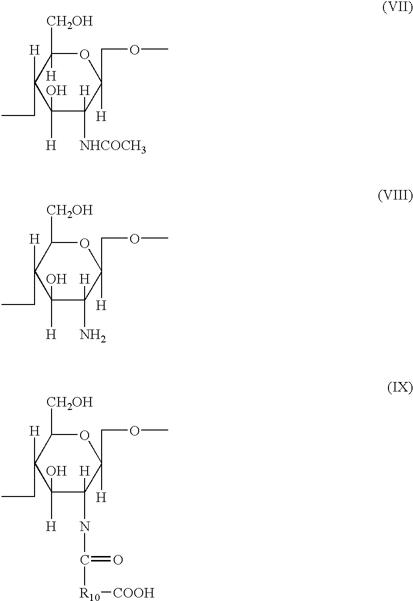
the unit (VII) being in an amount ranging from 0 to 30%, the unit (VIII) in an amount ranging from 5% to 50% and the unit (IX) in an amount ranging from 30% to 90%, wherein in the unit (IX), R₁₀ is chosen from radicals of formula: 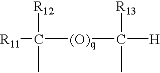
wherein q is equal to 0 or 1,
if q=0, R₁₁, R₁₂ and R₁₃, which may be identical or different, are chosen from a hydrogen atom, methyl, hydroxyl, acetoxy and amino residues, monoalkylamine residues and dialkylamine residues which are optionally interrupted by at least one nitrogen atom and/or optionally substituted with at least one group chosen from amine, hydroxyl, carboxyl, alkylthio and sulphonic groups, alkylthio residues in which the alkyl group bears an amino residue, at least one of the radicals R₁₁, R₁₂ and R₁₃ being, in this case, a hydrogen atom; or, if q=1, R₁₁, R₁₂ and R₁₃, which may be identical or different, are chosen from a hydrogen atom, and the salts formed by these compounds with bases or acids;
(6) polymers derived from the N-carboxyalkylation of chitosan;
(7) polymers corresponding to the general formula (X): 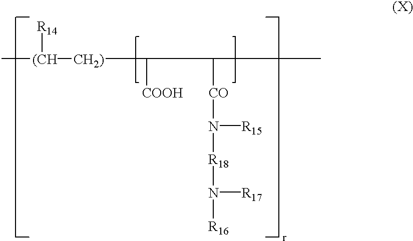
wherein R₁₄ is chosen from a hydrogen atom, and CH₃O, CH₃CH₂O and phenyl radicals,
R₁₅ is chosen from a hydrogen atom and lower alkyl radicals,
R₁₆ is chosen from a hydrogen atom and lower alkyl radicals,
R₁₇ is chosen from lower alkyl radicals and radicals corresponding to the formula:
—R₁₈—N(R₁₆)₂, wherein R₁₈ is chosen from —CH₂—CH₂—, —CH₂—CH₂—CH₂— and —CH₂—CH(CH₃)— groups, R₁₆ is chosen from hydrogen and lower alkyl radicals,
and the higher homologues of these radicals comprising up to 6 carbon atoms,
r is chosen such that the number-average molecular weight of the polymer ranges from 500 to 6,000,000;
(8) amphoteric polymers of the type -D-X-D-X— chosen from:
a) polymers obtained by the action of chloroacetic acid or sodium chloroacetate on compounds comprising at least one unit of formula:
-D-X-D-X-D- (XI)
wherein D is a radical 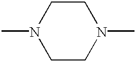
and X is chosen from the symbols of E and E′, wherein E and E′, which may be identical or different, are chosen from divalent alkylene radicals comprising at least one chain chosen from straight and branched chains comprising up to 7 carbon atoms in the main chain, wherein the main chain is optionally substituted with at least one hydroxyl group and E or E′ optionally comprises at least one atom chosen from oxygen, nitrogen and sulphur atoms, and 1 to 3 rings chosen from aromatic and heterocyclic rings; wherein the oxygen, nitrogen and sulphur atoms are present in the form of at least one group chosen from ether, thioether, sulphoxide, sulphone, sulphonium, alkylamine, alkenylamine, hydroxyl, benzylamine, amine oxide, quaternary ammonium, amide, imide, alcohol, ester and urethane groups;
b) polymers of formula:
-D-X-D-X— (XII)
wherein D is a radical 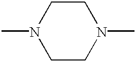
and X is chosen from the symbols E and E′ and wherein at least one X is chosen from E′ wherein E is chosen from divalent alkylene radicals comprising at least one chain chosen from straight and branched chains comprising up to 7 carbon atoms in the main chain, wherein the main chain is optionally substituted with at least one hydroxyl group and E optionally comprises at least one atom chosen from oxygen, nitrogen and sulphur atoms, and 1 to 3 rings chosen from aromatic and heterocyclic rings; wherein the oxygen, nitrogen and sulphur atoms are present in the form of at least one group chosen from ether, thioether, sulphoxide, sulphone, sulphonium, alkylamine, alkenylamine, hydroxyl, benzylamine, amine oxide, quaternary ammonium, amide, imide, alcohol, ester and urethane groups;
and E′ is chosen from divalent alkylene radicals comprising at least one chain chosen from straight and branched chains comprising up to 7 carbon atoms in the main chain, wherein the main chain is optionally substituted with at least one hydroxyl radical and the divalent alkylene radicals comprise at least one nitrogen atom, the at least one nitrogen atom being substituted with an alkyl chain, wherein the alkyl chain is optionally interrupted by an oxygen atom and wherein the alkyl chain comprises at least one functional group chosen from carboxyl functional groups and hydroxyl functional groups and wherein the alkyl chain is betainized by reaction with a reactant chosen from chloroacetic acids and sodium chloroacetate; and
(9) (C₁-C₅)alkyl vinyl ether/maleic anhydride copolymers partially modified by semiamidation with an N,N-dialkylaminoalkylamine or by semiesterification with an N,N-dialkanolamine.

28. The composition according to claim 27, wherein in (1) the at least one monomer derived from a vinyl compound bearing a carboxylic group is chosen from acrylic acids, methacrylic acids, maleic acids, and α-chloroacrylic acids.

29. The composition according to claim 27, wherein in (1) the at least one basic monomer derived from a substituted vinyl compound comprising at least one basic atom is chosen from dialkylaminoalkyl methacrylates, dialkylaminoalkyl acrylates, dialkylaminoalkylmethacrylamide and dialkylaminoalkylacrylamide.

30. The composition according to claim 27, wherein in (1) the dialkyl-diallylammonium salt is dimethyldiallylammonium chloride.

31. The composition according to claim 27, wherein in (2)(c) the at least one basic comonomer is chosen from esters comprising primary, secondary, tertiary and quaternary amine substituents of acrylic and methacrylic acids and the products of quaternization of dimethylaminoethyl methacrylate with dimethyl or diethyl sulphate.

32. The composition according to claim 27, wherein in (3) Z represents:
a) in an amount ranging from 60 to 100 mol %, the radical 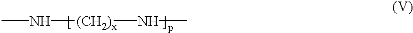
wherein x=2 and p=2 or 3, or x=3 and p=2
the radical being derived from a compound chosen from diethylenetriamine, triethylenetetraamine and dipropylenetriamine;
b) in an amount ranging from 0 to 40 mol %, the radical chosen from the radicals of formula (V) in which x=2 and p=1 derived from ethylenediamine, and the radicals derived from piperazine: 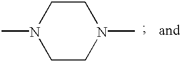
c) in an amount ranging from 0 to 20 mol %, the —NH—(CH₂)₆—NH— radical derived from hexamethylenediamine.

33. The composition according to claim 27, wherein in (4) the polymerizable unsaturated groups are chosen from acrylate, methacrylate, acrylamide and methacrylamide groups.

34. The composition according to claim 27, wherein in (6) the polymers derived from the N-carboxyalkylation of chitosan are chosen from N-carboxymethylchitosans and N-carboxybutylchitosans.

35. The composition according to claim 27, wherein in (7) the lower alkyl radicals are chosen from methyl radicals and ethyl radicals.

36. The composition according to claim 27, wherein in (9) the N,N-dialkylaminoalkylamine is N,N-dimethylaminopropylamine.

37. The composition according to claim 27, characterized in that said amphoteric polymers are chosen from copolymers of dimethyldiallylammonium salt and of acrylic acid.

38. The composition according to claim 1, further comprising at least one additional silicone other than polydialkylsiloxanes comprising dimethylsilanol end groups.

39. The composition according to claim 38, characterized in that the at least one additional silicone is chosen from non-volatile silicones chosen from the family of polyalkylsiloxanes comprising trimethylsilyl end groups and aminosilicones.

40. The composition according to claim 1, further comprising at least one agent that is beneficial for a keratin material, chosen from esters of C₁-C₃₀ carboxylic acids and of C₁-C₃₀ mono- and polyhydroxylated alcohols, plant, animal, mineral and synthetic oils, waxes, ceramides and pseudoceramides.

41. The composition according to claim 1, wherein the at least one copolymer of methacrylic acid and of a C₁-C₄ alkyl acrylate is in a concentration ranging from 0.01% to 20% by weight relative to the total weight of the composition.

42. The composition according to claim 41, wherein the at least one copolymer of methacrylic acid and of a C₁-C₄ alkyl acrylate is in a concentration ranging from 0.05% to 15% by weight relative to the total weight of the composition.

43. The composition according to claim 1, wherein the at least one silicone comprising dimethylsilanol end groups is in a concentration ranging from 0.001% to 20% by weight relative to the total weight of the composition.

44. The composition according to claim 43, wherein the at least one silicone comprising dimethylsilanol end groups is in a concentration ranging from 0.01% to 10% by weight relative to the total weight of the composition.

45. The composition according to claim 1, wherein the at least one polymer chosen from cationic and amphoteric polymers is in a concentration ranging from 0.001% to 20% by weight relative to the total weight of the composition.

46. The composition according to claim 45, wherein the at least one polymer chosen from cationic and amphoteric polymers is in a concentration ranging from 0.01% to 10% by weight relative to the total weight of the composition.

47. The composition according to claim 38, wherein the at least one additional silicone is in a concentration ranging from 0.001% to 20% by weight relative to the total weight of the composition.

48. The composition according to claim 47, wherein the at least one additional silicone is in a concentration ranging from 0.01% to 10% by weight relative to the total weight of the composition.

49. The composition according to claim 40, wherein the at least one agent that is beneficial for a keratin material is in a concentration ranging from 0.001% to 20% by weight relative to the total weight of the composition.

50. The composition according to claim 49, wherein the at least one agent that is beneficial for a keratin material is in a concentration ranging from 0.01% to 10% by weight relative to the total weight of the composition.

51. The composition according to claim 1, further comprising at least one surfactant chosen from anionic, nonionic, amphoteric and cationic surfactants.

52. The composition according to claim 51, wherein the at least one surfactant is chosen from anionic surfactants, mixtures of at least one anionic surfactant and at least one amphoteric surfactant, and mixtures of at least one anionic surfactant and at least one nonionic surfactant.

53. The composition according to claim 51, wherein the at least one surfactant is in a concentration ranging from 0.01% to 50% by weight relative to the total weight of the composition.

54. The composition according to claim 53, wherein the at least one surfactant is in a concentration ranging from 0.1% to 40% by weight relative to the total weight of the composition.

55. The composition according to claim 54, wherein the at least one surfactant is in a concentration ranging from 0.5% to 30% by weight relative to the total weight of the composition.

56. The composition according to claim 1, further comprising at least one additive chosen from thickeners, antidandruff agents, anti-seborrhoeic agents, fragrances, nacreous agents, hydroxy acids, electrolytes, fatty acid esters, preserving agents, silicone and non-silicone sunscreens, vitamins, provitamins, anionic and nonionic polymers, proteins, protein hydrolysates, and 18-methyleicosanoic acid.

57. The composition according to claim 56, wherein the provitamins are chosen from panthenol.

58. The composition according to claim 1, wherein the composition is in the form of a shampoo, a conditioner, a permanent-waving, straightening, dyeing or bleaching composition for hair, a rinse-out composition to be applied between the two steps of a permanent-waving or hair-straightening operation, or a washing composition for a body.

59. A composition for washing or caring for a keratin material comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups, said composition being effective for washing or caring for a keratin material.

60. A method for washing or caring for a keratin material, comprising applying to the keratin material a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

61. A process for treating a keratin material, comprising applying to the keratin material a cosmetic composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups,
and then optionally rinsing it out with water.

62. A composition comprising, in a cosmetically acceptable medium, one composition (1) comprising at least one copolymer chosen from crosslinked and non-crosslinked copolymers of methacrylic acid and of a C₁-C₄ alkyl acrylate, and another composition (2) comprising at least one polymer chosen from cationic and amphoteric polymers whose cationic charge density is greater than or equal to 3.5 meq/g and at least one silicone chosen from polydialkylsiloxanes comprising dimethylsilanol end groups.

63. A method of manufacturing a cosmetic composition, comprising including in the said composition at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g and at least one silicone chosen from polydialkylsiloxanes comprising dimethylsilanol end groups.

64. A composition to give hair sheen comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups, said composition being effective to give hair sheen.

65. A method to give hair sheen comprising applying to the hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

66. A composition to give hair lightness comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups, said composition being effective to give hair lightness.

67. A method to give hair lightness, comprising applying to the hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

68. A composition to give hair softness comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups, said composition being effective to give hair softness.

69. A method to give hair softness, comprising applying to the hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

70. A composition to give hair a smooth feel comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups, said composition being effective to give hair a smooth feel.

71. A method to give hair a smooth feel, comprising applying to the hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

72. A composition to give hair suppleness comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups, said composition being effective to give hair suppleness.

73. A method to give hair suppleness, comprising applying to the hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is greater than or equal to 3.5 meq/g, and at least one silicone chosen from polydialkylsiloxanes comprising dimethylsilanol end groups.

74. The composition according to claim 22, wherein A in (9) is —CH₂—CH₂—O—CH₂—CH₂—.

75. The process according to claim 61, wherein the keratin material is hair.
